(19) United States
(12) Patent Application Publication
(10) Pub. No.: US 2012/0101405 A1
(43) Pub. Date: Apr. 26, 2012

(54) METHOD FOR PREDICTING CUTANEOUS AFFERENT NERVE FIBER EXCITATION

(75) Inventors: Ole Kæseler Andersen, Skorping (DK); Kristian Hennings, Aalborg SV (DK); Carsten Dahl Mørch, Aalborg (DK)

(73) Assignee: AALBORG UNIVERSITET, Aalborg O (DK)

(21) Appl. No.: 13/277,048

(22) Filed: Oct. 19, 2011

Related U.S. Application Data
(60) Provisional application No. 61/405,358, filed on Oct. 21, 2010.

(51) Int. Cl. A61B 5/05

(52) U.S. Cl. 600/554

(57) ABSTRACT

Aspects of the invention relate to a device for predicting cutaneous afferent nerve fiber excitation from an electrical potential applied to a cutaneous tissue by an electrode and methods of using the same. In some embodiments, such devices and methods concern the electrical stimulation of nerves in the skin so as to ameliorate pain in a subject. Accordingly, methods making and using cutaneous electrodes are provided.

### CROSS-REFERENCE TO RELATED APPLICATIONS

This application claims the benefit of priority to U.S. Provisional Patent Application No. 61/405,358, filed on Oct. 21, 2010, the disclosure of which is hereby expressly incorporated by reference in its entirety.

### BACKGROUND OF THE INVENTION

1. Field of the Invention

The invention relates to the field of biomedical technologies. More specifically, the invention relates to the field of electrical stimulation of nerves in the skin, such as electrical stimulation for relief of pain. More specifically, the invention provides a method and a device for predicting cutaneous afferent nerve fiber excitation from an electrical potential applied to a cutaneous tissue by an electrode, and a method for designing cutaneous electrodes.

2. Description of the Related Art

In several biomedical technologies, both for research and clinical treatment, electrical stimulation is applied in order to activate nerve fibers. One such technology is trans cutaneous electrical stimulation where electrodes are placed at the surface of the skin and repeated electrical pulses are applied to obtain pain relief, e.g. In chronic low back pain patients. In this application it is important that the stimulation is not painful. This is attained by applying the electrical stimulation through large electrodes. To understand why large electrodes are needed in order to avoid pain sensation one must understand how the current flows through the skin, the morphology of the nerve fibers, and how the nerve fibers are excited by current flow.

Several theoretical models have been proposed for describing the nerve fibers innervating the skin. However, such models have not been able to precisely predict the electrical behavior of the complicated nerve structures in the skin, and thus the methods are not able to correctly predict the performance of surface electrodes or electrodes for implanting into the skin layers. Particularly, the models are not suited for predicting selective stimulation of the different types of afferent nerve fibers in the skin. Therefore, to design stimulation electrodes, a large number of tests on persons are required to obtain an electrode with desired properties. This is a time consuming and very expensive process, since a given electrode configuration needs to be manufactured before its properties can be precisely tested.

### SUMMARY OF THE INVENTION

Thus, there is a need for more precise tool for predicting electrical behavior of skin electrodes for stimulating nerve fibers in the skin at an early state in the design phase.

In a first aspect, the invention provides A method for predicting afferent nerve fiber excitation from an electrical potential applied to a cutaneous tissue by an electrode, the method comprising

providing a model of propagation of electrical potentials in the cutaneous tissue,

providing a model of geometric distribution of afferent nerve fibers in the cutaneous tissue, wherein said model comprises a stochastic model of branching of afferent nerve fibers in the cutaneous tissue,

providing a geometric representation of the electrode in or on a surface of the cutaneous tissue, and

predicting afferent nerve fiber excitation from the electrical potential applied to the electrode by means of combining said model of propagation, said model of geometric distribution of afferent nerve fibers, and said geometric representation of the electrode.

This method takes into account the branching of nerve fibers in the skin layers, namely by modeling this branching stochastically, i.e. By including one or more random factors regarding the nerve fiber branching behavior, thus more correctly arriving at a more realistic model of the location of nerves in the skin. The stochastic modeling of nerve fiber branching may include introducing a probability that the nerve fiber will branch at each node of ranvier into two new branches, and that the two new branches will continue in a stochastic direction, but towards the surface of the skin, and that nerve fibers will terminate at a random skin depth. Hereby, a more precise prediction of the nerve fiber excitation by a given electrode geometry is obtained. Furthermore, the model can be implemented in computer software, thus allowing easy testing of electrode configurations at an early design state without involving expensive and time consuming tests on humans.

The method is useful in designing electrodes if it is desirable to selectively activate certain nerve fibers e.g. activating tactile fibers, such as touch sensitive fibers, without activating pain mediating fibers or the opposite (i.e. activation of pain inducing fibers without tactile activation).

In one embodiment, the stochastic model of branching of afferent nerve fibers comprises specifying a probability that a nerve fiber will branch into first and second branching nerve fibers, such as a probability of 1-20%, such as a probability of 10%, that the nerve fiber will branch at a given node. In such an embodiment, the model may comprise specifying a first stochastic direction towards a skin surface for the first branching fiber and a second stochastic direction towards a skin surface for the second branching nerve fiber. In such an embodiment, said first and second directions towards a skin surface may be determined by respective uniform distributions of azimuthal and horizontal angles towards the skin surface. A location in the cutaneous tissue where a nerve fiber emerges may be modeled as a random location within a specified area. A cutaneous depth where a nerve fiber ends may be modeled stochastically.

The model of propagation of electrical potentional in the cutaneous tissue preferably comprises at least two different cutaneous layers with different electrical properties. Especially, this model comprises modeling of different electrical properties of the cutaneous layers: hypodermis, dermis, epidermis, and stratum corneum. The model of propagation of electrical potentials in the cutaneous tissue may comprise a Finite Element model, more specifically such a Finite Element model may be a two dimensional axial symmetrical Finite Element model. The model of propagation of electrical potentials in the cutaneous tissue may comprise a model of extracellular potentials.

The method may comprise separate models of geometric distribution in the cutaneous tissue of a nociceptive type of nerve fiber and a non-nociceptive type of nerve fiber, with respective different stochastic parameters specified for said two types of nerve fibers. Especially, such a method may comprise predicting separate non-nociceptive nerve fiber excitation and nociceptive nerve fiber excitation from the electrical potential applied to the electrode. More particularly, the method may comprise providing separate models of distribution in the cutaneous tissue of Aβ nerve fibers and M nerve fibers.

The method may comprise calculating a membrane potential along points representing nodes of Ranvier on the nerve fibers. Especially, a current equilibrium is calculated at each node of Ranvier according to Kirchoff's law.

In a second aspect, the invention provides a computer executable program code arranged to cause a computer device to perform the method according to the first aspect.

In a third aspect, the invention provides a device arranged to perform the method according to claim 1, e.g. a Personal Computer or a server computer or work station or the like.

In a fourth aspect, the invention provides a method for designing a cutaneous electrode, the design method comprising

performing the method according to the first aspect, and

changing a geometric design of the cutaneous electrode based on the predicted afferent nerve fiber excitation.

In a fifth aspect, the invention provides an electrode for cutaneous stimulation designed according to the method defined in the fourth aspect.

It is to be understood that the advantages and embodiments mentioned for the first aspects, apply as well for the second, third, fourth, and fifth aspects.

### BRIEF DESCRIPTION OF THE DRAWINGS

Embodiments of the invention will be described, by way of example only, with reference to the drawings, in which

FIG. 1 illustrates in block diagram form basic elements of a method according to the invention,

FIG. 2 illustrates the grid of a preferred two-dimensional axis symmetrical Finite Element model of the four upper skin layers,

FIG. 3 illustrates an example of the stochastic nerve fiber branching mode,

FIG. 4 shows graphs of current density in the skin modeled by the Finite Element model,

FIG. 5 shows graphs indicating experimental electrode-skin impedances and the electrode-skin impedance predicted by the Finite Element model,

FIG. 6 shows graphs indicating results of an experiment where perception threshold was determined for different electrode diameters and compared with predicted nerve fiber excitation,

FIG. 7 shows graphs indicating for different results from an experiment where subjects were asked to score a pain sensation felt with different electrode sizes using different pain descriptors,

FIG. 8 shows prediction results for excitation thresholds of nerve fibers in case of two different stratum corneum thicknesses.

### DETAILED DESCRIPTION OF PREFERRED EMBODIMENTS

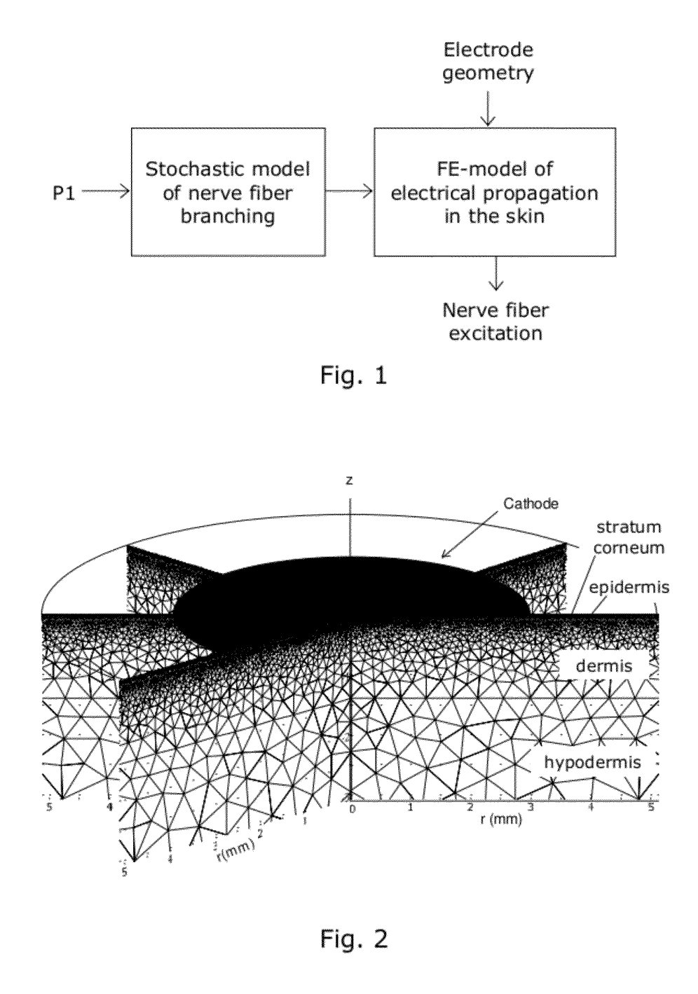

FIG. 1 shows basic elements of an embodiment for a prediction method for predicting nerve fiber excitation. A Finite element model of electrical propagation of an electrical potential in the skin is provided, preferably a model taking into account different properties for several layers of the skin. However, other types of models than Finite Element models may be used. A stochastic model of nerve fiber branching is used as input to the electrical skin propagation model in order to provide input regarding location of nerve fibers in the skin. At least one, preferably more probability parameters P1 are input to this stochastic nerve fiber branching model. Such a parameter P1 may be the probability that a nerve fiber branches at a given node. If branching occurs, the direction towards skin surface for the two new branches may then be described by a probability density function. Furthermore, the emerging location for a nerve fiber and the skin depth where a nerve fiber ends may likewise be stochastically modeled.

A geometric representation of a stimulation electrode is also input to the Finite Element model. Such geometric electrode input may especially include size and position of the electrode, i.e. whether the electrode is placed on a surface of the skin or implanted into a given layer of the skin.

From the described inputs, the combined Finite Element and nerve fiber model can then calculate a predicted nerve fiber excitation which can be expected with the given electrode configuration. Especially, it may be desired to predict respective predicted excitations from different afferent nerve fiber types, such as Aδ and Aβ nerve fiber excitations.

In the following, a specific implementation example of the invention will be described. Experiments serving to verify the method according to the invention are also described. Furthermore, predictions and experiments with different electrode sizes are described. References referred to are indicated by [X], and a list of references can be found in the back of the present chapter.

The FE model described four horizontal layers; stratum corneum, epidermis, dermis, and hypodermal used to estimate the excitation threshold of Aβ-fibers terminating in dermis and Aδ-fibers terminating in epidermis. The perception thresholds of 11 electrodes with diameters ranging from 0.2 mm to 20 mm were modeled and assessed on the volar forearm of healthy human volunteers by an adaptive two-alternative forced choice algorithm.

The model showed that the magnitude of the current density was highest for smaller electrodes and decreased through the skin. The excitation thresholds of the Aδ-fibers were lower than the excitation thresholds of Aβ-fibers when current was applied through small, but not large electrodes. The experimentally assessed perception threshold followed the lowest excitation threshold of the modeled fibers. The model confirms that preferential excitation of Aδ-fibers may be achieved by small electrode stimulation due to higher current density in the dermoepidermal junction.

### Introduction

In experimental and clinical pain studies, painful stimuli are often applied in order to probe the state of the pain system. Such painful stimuli can be applied by cutaneous electrical stimulation which is easy to control and safe to apply. However, electrical stimulation is a non-natural stimulation modality as the action potential is provoked by an electrical potential difference across the nerve fiber membrane. In contrast, natural stimuli such as pressure or temperature changes are mediated through specialized ion channels or receptor organs, so that afferents are selectively activated by one or a few stimulus modalities. The excitation of cutaneous fibers is therefore governed by the electrical and β geometrical properties of the fiber, surrounding tissue, and the external electrical potential but not by receptor properties. Consequently, both non-nociceptive large-diameter myelinated Aβ fibers, thinly myelinated Aδ fibers and unmyelinated C-fibers may be activated by the electrical stimulation [1]. The excitation threshold of non-nociceptive fibers is generally lower than the excitation threshold of nociceptive fibers [2]. Non-nociceptive fibers will therefore also be activated when activating nociceptive fibers. This leads to a mixed afferent input of both non-nociceptive and nociceptive information to the central nervous system which also explains the artificial sensation perceived. This may cause an attenuation of the nociceptive information due to the gate-control mechanism [3].

Intracutaneous electrical stimulation was introduced by Bromm and Meier [4] to achieve activation of nociceptors without activating non-nociceptive fibers, or at least to activate the non-nociceptive fibers to a less extent. To overcome the disadvantages from this invasive intracutaneous method, alternative electrode configurations have been introduced as either an array of small diameter surface electrodes [5], or single small diameter electrodes [6] sometimes with a large concentric anode [7]. Kaube et al. suggested that a higher current density at the dermoepidermal junction may be the cause of a more preferential activation of nociceptors when using small electrode surfaces [7]. The current density in the skin cannot be measured, so to test the hypothesis a theoretical model is needed.

The excitation of a single straight nerve fiber branch has been described [8]. However, cutaneous sensory fibers show substantial branching [9, 10]and there are large differences in the morphology between Aδ-fibers and Aβ-fibers [11]. The depth of the fiber endings may be particularly important for the electrically evoked excitation of the nerve fibers as the Aβ-fibers terminate in the dermis [9] whereas the Aδ-fibers penetrate into the vial layers of epidermis [12]. In McNeal's nerve fiber excitation model, the nerve fiber was subject to a single point source although McNeal suggested several more realistic models for describing a more realistic external electrical potential. The electrical potential in the skin can be described by finite element (FE) models describing inhomogenous and anisotropic properties of the skin [13, 14].

This study tested the hypothesis that high current density at the dermoepidermal junction could be generated by small area electrodes and preferentially activate Aδ-fibers and to a lesser extent Aβ-fibers thus generating a sharp, pricking or cutting sensation. The aim of the study was to model the current density and electrical field in the skin by a FE model to describe the action potential generating extracellular potential. Moreover, the nerve excitation thresholds were calculated using a stochastically branching nerve fiber model. A validation of the modeled excitation thresholds was performed by experiential assessment of perception thresholds for varying electrode diameters in humans.

### Method for the Experiment, Finite Element Model of the Electric Potential at the Volar Forearm

A two-dimensional axial symmetrical FE model of the skin was created to simulate the extracellular potential (Comsol Multiphysics 3.5a, Sweden). The axial symmetry line was vertical; the horizontal distance from the symmetry line was denoted r, and the vertical axis, z, was zero at the skin surface and decreased through the skin layers.

FIG. 2 illustrates a segment of the geometry of the rotational symmetrical FE model. The FE model comprised four horizontal layers: stratum corneum, epidermis, dermis, and hypodermal layers. These layers were modeled as horizontal layers and material properties were adopted from literature observations, see the Table below. The FE model was constructed as a two dimensional model with axial symmetry around z-axis. An example of a cathode with a diameter of 6 mm is depicted. A mesh was generated to describe the current density and electric potential in the skin. The mesh was densest in the stratum corneum below the cathode. Please note that the figure only depicts a segment of the 5 cm wide and 6389 μm deep FE model.

| | Electrical | Refer- | Thickness | Refer- |
|---|---|---|---|---|
| Layer | conductivity (S/m) | ences | (μm) | ences |
| Stratum | 2.0 × 10⁻⁵ [10; 43] | [23, 25] | 29 [26; 34; 43] | [40, 41] |
| Corneum | | | | [23] |
| Epidermis | Horizontal: 0.95, | [24, 25] | 60 [26; 34] | [40, 41] |
| | Vertical: 0.15 | | | |
| | [10; 37] | | | |
| Dermis | Horizontal: 2.57, | [24, 25] | 1300 [7; 21] | [42, 43] |
| | Vertical: 1.62 | | | |
| | [10; 37] | | | |
| Hypo- | 2.0 × 10⁻² [8; 9] | [44] (fat) | 5000 | |
| dermis | | [45] | | |
| | | (muscle) | | |

The electrical potential was modeled in a stationary conductive media by reducing Ohm's and Gauss' laws to:

−∇·(σ∇V_{FE}−Jₑₓₜ)=0 Equation 1

where σ was the conductivity, V_{FE} was the electrical potential calculated by the FE model, Jₑₓₜ was the externally generated current density.

Stimulation was simulated as an inward current through the center part of the upper boundary modeling the surface of the skin. The anode was simulated as a boundary condition for entire the lower boundary of the hypodermis in order to avoid variables such as size of or distance to e.g. a concentric electrode. All other external boundaries were considered as electrically insulated. A multi resolution tetrahedral mesh was generated for solving the model. 100 mesh points were generated at the boundary, modeling the cathode and 1000 mesh points were generated at the remaining upper surface of the model. The mesh points were densest at the edge of the cathode boundary where the inward current was expected to be discontinuous. A convergence study was conducted to ensure sufficient mesh density and the radial extent if the model. The electrical potential did not change by refining the mesh beyond 30000 elements. Furthermore, there were no changes in the electrical potential in the dermoepidermal junction by increasing the radial extent of the model beyond 5 cm[13]. Therefore, the FE model consisted of 30000 elements and had a radial extent of 5 cm. Solving the FE model lasted 36 seconds on a quad-core, 8 GB ram PC using Comsol Multiphysics 3.5a, Sweden.

### Stochastic Model of Nerve Fiber

2300 Aβ fibers and 1000 Aδ fibers were modeled as single fibers emerging from a random location in a 10 by 10 mm area around the center of the FE model at the lower boundary. The number of fiber-branches entering the model was selected so that the densities of the Aβ- and Aδ- nerve endings were approximately 59.0 and 163 per mm², respectively [10, 15].

The fibers followed a straight line with a random angle (horizontal and azimuthal angles were uniformly distributed in the interval from 0 to π/10 and 0 to 2π, respectively) from the hypodermis towards the surface of the skin.

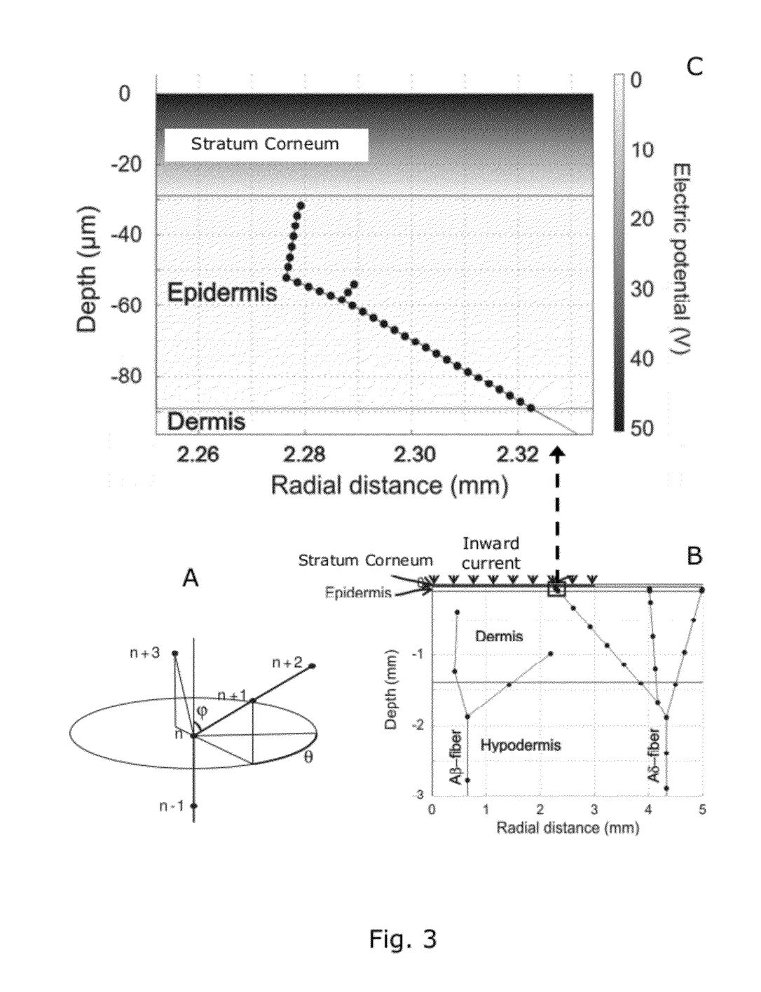

FIG. 3 illustrates examples on stochastic models of nerve fibers. (A) An example of a nerve fiber branching at the n-th node of Ranvier. The location of the next node (n+1) was obtained by randomly selecting the horizontal angle (θ) and azimuthal angle (φ) were uniformly distributed in the interval from 0 to π/10 and 0 to 2π. Additional nodes (e.g. node n+2) could be added in a straight line if the fiber was not terminated. In the same way another branch was added to the n-th node. Here only the node n+3 is shown. (B) An example of an Aβ-fiber (left) with one branch point and an Aδ-fiber (right) with multiple branch points are depicted in this subsection of the model. All Aβ-fibers terminated in the dermis whereas the Aδ-fibers terminated in the epidermis. Each solid circle represents a node of Ranvier. The electrodes (diameter of 6 mm in this example) were simulated as an inward current and is indicated as arrows. The box at the left-most branch of the Aδ-fiber in B is enhanced in C. (C) The Aδ-fibers were modeled as unmyelinated in the epidermal layer which manifests as a dense distribution of nodes of Ranvier. The gray scale colors indicate the extracellular potential calculated by the FE model for an electrode size of 6 mm.

Branching of the nerve fibers occurred at each node of Ranvier after entering dermis with a probability assumed to be 10%. The ratio of internodal space to fiber diameter and the ratio of axon and fiber diameters were adopted from McNeal [8] to be 100 and 0.7, respectively. The nodel length was modeled as 4 μm [9]. The initial diameter of the Aβ fibers was modeled as 9 μm but shrunk at each branch point with a factor assumed to be 0.98. The Aβ-fibers terminated at a random but uniformly distributed depth in the dermis [16]. The initial diameter of the Aβ fibers was modeled as 4 μm but shrunk at each branch point with a factor assumed to be 0.95. The diameter of the Aδ-fibers was reduced to 1 μm as the nerve entered epidermis and the inter nodal distance was decreased to the nodal length (4 μm) to model the depletion of myelin sheets in the epidermis [17]. The Aδ fibers terminated at a random but uniformly distributed depth in the vial epidermis [12].

The membrane potential was calculated along points representing nodes of Ranvier on the simulated nerve fibers. The sections between these nodes were considered to be non-conducting mimicking the electrical properties of myelin sheets. The current equilibrium was calculated at each node by Kirchoff's law stating that the membrane current at a node is the sum of the capacitive and ionic membrane currents which equals the sum of the connecting axial currents.

$${C}_{m , n} \frac{d ( {V}_{i , n} - {V}_{e , n} - {V}_{r} )}{d t} + {G}_{m , n} ( {V}_{i , n} - {V}_{e , n} - {V}_{r} ) = {\sum }_{k = 1}^{K} {G}_{a , nk} ( {V}_{i , k} - {V}_{i , n} ) \qquad Equation 2$$

where C_{m,n} is the membrane capacitance at the node n. V_{i,n}, V_{e,n}, and Vᵣ are the intra-cellular, extracellular and resting membrane potentials, receptively so that (V_{i,n}−V_{e,n}−Vᵣ) is the reduced trans-membrane potential, V_{n,n}. G_{m,n} is the membrane conductance, K is the number of neighboring nodes k, and G_{a,nk} is the intra-axonal conductance between node n and k.

These model parameters were adopted from McNeal [8]. By inserting V_{n,n} at node n and k and rearranging, Equation 2 reduces to:

$$\frac{d {V}_{n , n}}{d t} = - \frac{{G}_{m , n}}{{C}_{m , n}} {V}_{n , n} + {\sum }_{k = 1}^{K} \frac{{G}_{a , nk}}{{C}_{m , n}} ( {V}_{n , k} - {V}_{n , n} ) + {\sum }_{k = 1}^{K} \frac{{G}_{a , nk}}{{C}_{m , n}} ( {V}_{e , k} - {V}_{e , n} ) \qquad Equation 3$$

This can conveniently be expressed on matrix form:

\dot{V}ₙ=AVₙ+B Equation 4

The matrix A in Equation 4 is given as:

$$A = [ \begin{matrix}- \frac{1}{{C}_{m , 1}} ( {G}_{m , 1} + {\sum }_{k = 1}^{K} {G}_{a , 1 k} ) & … & {w}_{1 N} \frac{{G}_{a , 1 N}}{{C}_{m , 1}} \\ ⋮ & ⋱ & ⋮ \\ {w}_{N 1} \frac{{G}_{a , 1 N}}{{C}_{m , N}} & … & - \frac{1}{{C}_{m , N}} ( {G}_{m , N} + {\sum }_{k = 1}^{K} {G}_{a , Nk} )\end{matrix} ]$$

The weights w_{n,k} is one if there is a connection from node n to k, and zero otherwise (please note that per definition wₙₖ=Wₖₙ). The vector B in Equation 4 is given as:

$$B = [ \begin{matrix}{\sum }_{k = 1}^{K} \frac{{G}_{a , 1 k}}{{C}_{m , 1}} ( {V}_{e , k} - {V}_{e , 1} ) \\ {\sum }_{k = 1}^{K} \frac{{G}_{a , Nk}}{{C}_{m , N}} ( {V}_{e , k} - {V}_{e , N} )\end{matrix} ]$$

A solution to Equation 4 is:

Vₙ=c₁e^{At}−A⁻¹B Equation 5

Since the reduced trans-membrane potential (Vₙ) is zero at time zero the coefficient c₁ can be found to be A⁻¹B and consequently the electrical potential can be described as:

Vₙ=A⁻¹(−B+e^{At}B) Equation 6

The electrical properties of the nerve were assumed to be constant at all nodes [8].

The extracellular potential at the n′th node with the position (x,y,z), V_{e,n}, is taken from the potential calculated by the FE model so that V_{e,n} (x,y,z)=V_{FE}(r,z), where r=\sqrt{jx²+y²}. It was assumed that the electrical properties and geometry of the nerve did not influence V_{FE}.

### Estimating the Perception Threshold

Excitation of a single cutaneous sensory fiber only leads to perception of stimulation when stimulating the most sensitive parts of the hand, but more sensory fibers need to be excited in order to evoke perception of the cutaneous stimulus [18]. For comparison with the experimentally assessed detection thresholds, the modeled perception thresholds were therefore approximated as the mean excitation thresholds of the 3% of the simulated fibers with the lowest threshold. Furthermore, a series of models were performed by considering perception after excitation of 0.1%, 0.3%, 1%, 3%, 10%, 30%, and 100% of the simulated fibers.

### Thickness of Stratum Corneum

The thickness of the stratum corneum was systematically modeled from 29 μm to 0.1 mm, 0.3 mm and 1.0 mm to investigate the feasibility of preferential activation of nociceptive fibers at sites with thick layers of cornified skin (e.g. heel area or palm of the hand).

### Experimental Assessment of Perception Threshold

Fourteen healthy volunteers (10 male, 4 female) between the ages of 19 and 28 years participated in the study. All subjects gave written, informed consent acknowledging that they would experience experimental painful stimuli and that they were free to withdraw from the experiment at any time without prejudice. All of the procedures were approved by local ethics committee (ref. no N-20070027).

A circular stainless steel cathode was placed on the volar forearm 10 cm from the elbow joint. Eleven electrodes with diameters of 0.2 mm, 0.9 mm, 1.5 mm, 2.0 mm, 4.0 mm, 6.0 mm, 8.0 mm, 10.0 mm, 12.0 mm, 16.0 mm, or 20.0 mm were used. The stainless steel electrode protruded 0.2 mm from a plastic base having an outer diameter of 22 mm for all electrode sizes. All electrodes were dry, i.e. no electrode gel was used. A 5 cm×9 cm anode (Pals, Axelgaard, Calif., USA) was placed on the dorsal side of the forearm. A slight pressure was applied to the electrode by a band around the arm. The electrodes were tested in random order. The subject was sitting in front of a computer screen and resting the forearm on a table. The impedance of each electrode was measured between 1 Hz and 10 kHz, by applying a stochastic signal with a magnitude of 1 μA for 30 sec [19].

A 1 ms constant current pulse was used to assess the perception threshold. The intensity of the initial stimulation was manually set to an intensity that was weak but perceivable. An adaptive two-alternative forced-choice task [20] was implemented on a PC to assess the perception threshold (LabView, National Instruments, Austin, Tex., USA). The screen in front of the subject showed two squares. The squares lit up one at a time and stimulation was applied when one of two squares lit. The subject was asked to indicate which square was lit when the stimulation was applied. The current of the stimulation pulse was increased by 30% if the answer was incorrect. The current of the stimulation pulse was decreased by 30% when three correct answers were provided. The three correct answers did not need to be consecutive so incorrect answers and consequent increases could be intermixed [21]. Fifty pulses were applied for each electrode size. In this task, the subjects would provide answer at random when the stimulation was not perceived and thus provide a correct answer in half of the trials. The provided answers were therefore fitted to an S-curve that was constrained between probabilities of 0.5 to 1 (Equation 7). The perception threshold was defined as the current corresponding to 75% correct answers.

$$P = \frac{1}{2} + \frac{1}{2 ( 1 + A ( B - I ) )} \qquad Equation 7$$

P is the probability of a correct answer, I is the current of the stimulation pulse, B is the fitting parameter indicating the current at which the probability a correct answers is 75%, i.e. the perception threshold. A is the fitting parameter indicating the slope of the fitted curve at B. The experimentally assessed perception thresholds were compared to the simulated excitation thresholds of the Aβ- and Aδ-fibers.

A single 1 ms stimulation of 10 times the perception threshold was applied after assessing the perception threshold, so that stimuli could be perceived as painful and the pain quality could be assessed. The subjects were asked to describe the quality of the sensation in a McGill pain questionnaire [21]. The McGill pain questionnaire contains 20 groups of words with two to six words in each group. The subjects were allowed to select only one word from each group, if one of the words described the sensation of the stimulation. The questionnaire was provided in a Danish version for subjects preferring that [22].

### Data Analysis

Linear regression was performed to investigate the effect of the electrode size on the impedance. A multinomial logistic regression test was performed for each category of pain descriptors to investigate the influence of electrode size on pain quality. Results are provided as mean±standard error of the mean. A significance level of P<0.05 was considered statistically significant.

### Results

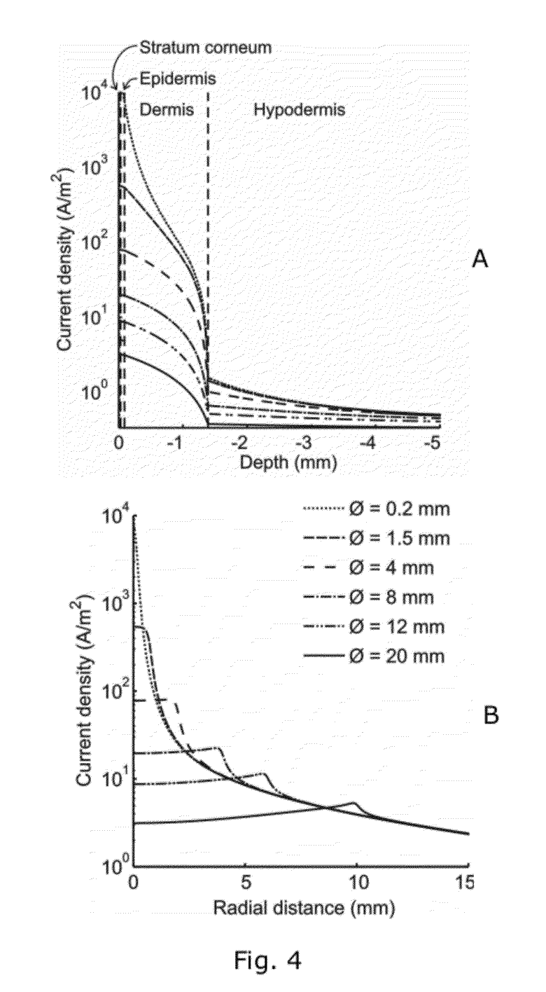

The magnitude of the current density FIG. 4 illustrates current density modeled by a finite element model. (A) The magnitude of the current density decreased uniformly below the cathode (z=0) towards the anode. Dashed vertical lines indicate transitions between skin layers. The decrease was steepest immediately above the hypodermal layer (z=−1.289 mm). (B) The magnitude of the current density at the dermoepidermal junction (z=89 μm i.e. reflecting a modeled stratum corneum thickness of 29 μm) was highest at the center of the cathode for electrode diameters (Ø) smaller than 4 mm, whereas the magnitude of the current density was largest at the edge of the electrode for larger electrodes.

The electric potential, as seen in FIG. 3, and the magnitude of the current density of the skin, seen in FIG. 4, were calculated by the FE model and used to estimate the fiber excitation of a stochastically branching nerve model. The FE model showed that the magnitude of the current density was highest for smaller electrodes, as expected and that the magnitude of the current density was highest immediately below the electrode (r=0) and decreased further below the electrode and with a marked decrease in the lower parts of the dermal layer (FIG. 4A). Probing the magnitude of the current density at the depth of the dermoepidermal junction showed a maximal current density at the edge of electrodes for electrodes with a diameter larger than 4 mm. Otherwise the magnitude of the current density had a maximum at the center of the electrode (r=0) and decreased with distance from the electrode (FIG. 4B).

### Resistance

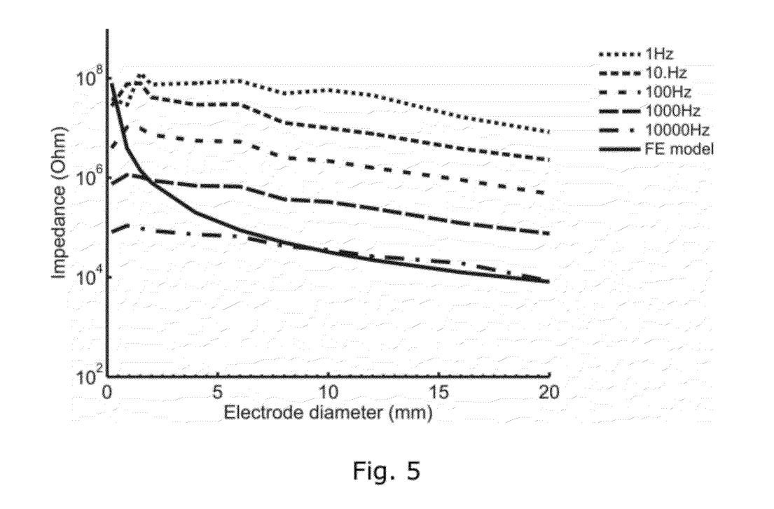

FIG. 5 illustrates electrode-skin impedance. The experimentally assessed impedance generally decreased as the electrode size increased, except for the smallest electrode (less than 0.9 mm). The error bars indicate the 95% confidence intervals.

The measured impedances of the electrodes were generally higher for the small electrodes (e.g. 1100 kΩ for 0.9 mm compared to 75 kΩ for 20 mm electrodes at 1 kHz; FIG. 4B). However, the smallest electrode (0.2 mm diameter) exhibited lower impedance (747 kΩ at 1 kHz) than other small electrodes (0.9 mm: 1143 kΩ; 1.5 mm: 1038 kΩ; 2.0 mm: 882 kΩ). The resistances estimated by the FE model decreased as electrode size increased (P<0.001). The modeled resistances were similar to the impedance measured at 10 kHz for electrodes with diameters larger than 6 mm.

### Branching Nerve Model

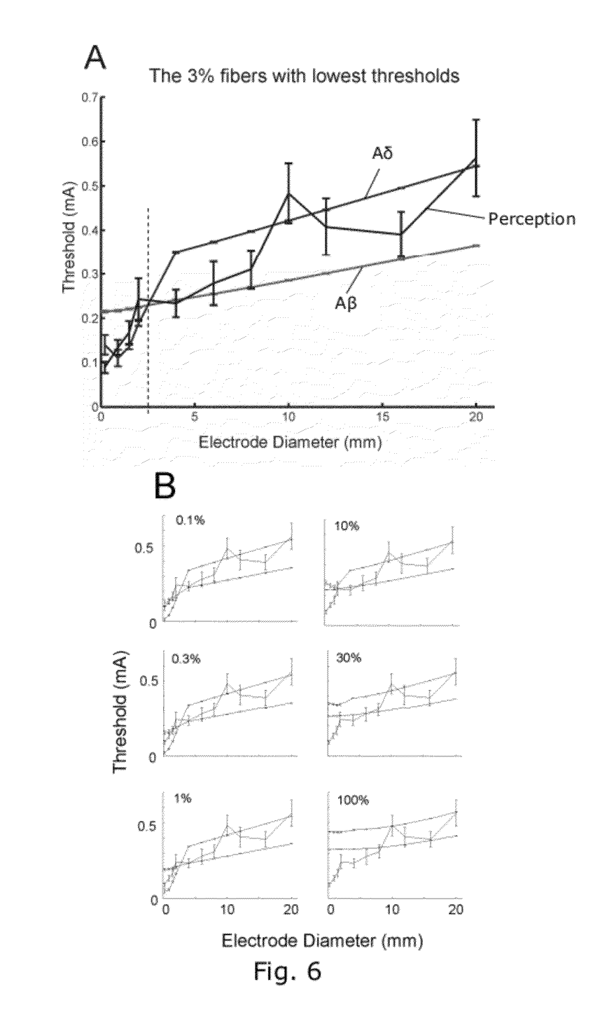

FIG. 6 illustrates results of perception threshold. (A) The experimental perception threshold was assessed by stimulation through electrode with different sizes. The experimental perception threshold followed the simulated excitation threshold of Aδ-fibers for electrodes smaller than 3 mm whereas the variation in the perception threshold makes it more ambiguous which fiber type was activated for electrodes larger than 3 mm, assuming that 3% of the afferent fibers are needed to evoke a perception of the stimulus. (B) The simulated perception threshold of the Aδ-fibers was lower than the simulated perception threshold of the Aβ-fibers for electrodes smaller than 3 mm if it was assumed that perception can be evoked by excitation of less than 10% of the fibers.

The stochastic branching of the modeled nerve fibers resulted in a density of 59±0.1 Aβ-fiber endings pr.mm² terminating at a depth of 916±5 μm and 153±1.2 Aδ-fiber endings pr.mm² terminating at a depth of 59.5±0.2 μm.

The excitation thresholds of the 3% of the Aδ-fibers with the lowest threshold were lower than excitation thresholds of the 3% of the Aβ-fibers with the lowest threshold for electrodes with diameters less than 3 mm. The thresholds of the Aδ-fibers increased more rapidly than the thresholds of the Aβ-fibers for increasing electrode diameter. Moreover, for electrode sizes larger than 3 mm, the excitation thresholds were lowest for Aβ-fibers.

### Pain Quality

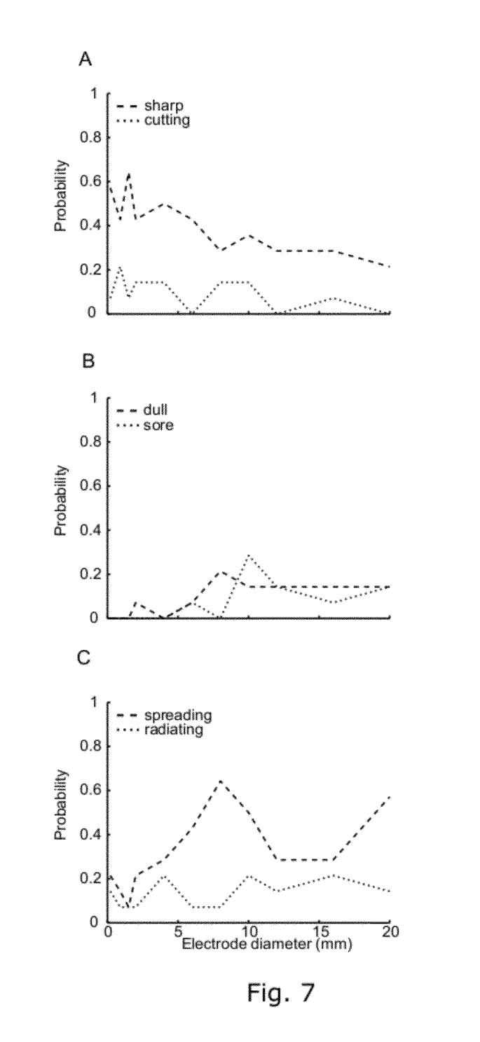

FIG. 7 illustrates experimental results for different pain descriptors and different electrode sizes. The subjects filled-in a McGill pain questionnaire after a single electrical stimulation by 11 different electrode sizes at 10 times perception threshold. Six of the pain descriptors had a statistically significant association with the electrode size (p<0.05; multinomial logistic regression) and are shown here; ‘sharp’ and ‘cutting’ were used more often for small electrodes (A), whereas ‘dull’, ‘sore’, ‘spreading’, and ‘radiation’ were used more often for large electrodes (B and C).

The words ‘sharp’ and ‘cutting’ were used more often for smaller electrodes than larger, whereas the words ‘dull’, ‘sore’, ‘spreading’, and ‘radiating’ were used more often for larger electrodes than smaller (p<0.05; multinomial logistic regression).

### Thickness of Stratum Corneum

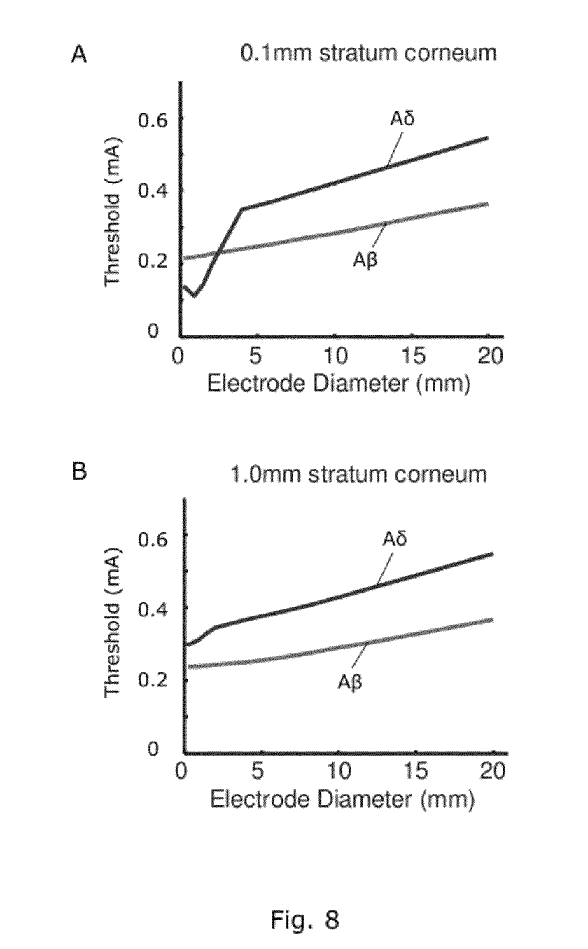

FIG. 8 illustrates the thickness of the stratum corneum was increased to from 29 μm to 1 mm in a series of simulations Two examples of excitation thresholds of Aδ- and Aβ-fibers are shown in skin with a stratum corneum thicknesses of 0.1 mm and 1.0 mm. The simulations showed that the excitation of Aδ-fibers increased above the excitation threshold of Aβ-fibers also for small electrodes if the thickness of the stratum corneum was larger than 0.8 mm.

When modeling electrical stimulation through small electrodes, the excitation thresholds of the Aδ-fibers increased more than the excitation thresholds of the Aβ-fibers. Therefore, the excitation threshold of the Aδ-fibers increased above the excitation threshold of the Aβ-fibers when the thickness of the stratum corneum was higher than 0.8 mm.

### Discussion

Preferential excitation of cutaneous Aδ-fiber by small area electrodes is possible. Evidently, a numerical description of the extracellular potential with a FE model and modeling the excitation threshold for stochastically branching nerve fibers revealed preferential excitation of Aδ-fiber for circular electrodes with diameters lower than 4 mm. However, in areas with thick cornified skin layers, preferential excitation of Aδ-fibers does not seem feasible. This finding was validated by assessing the perception threshold and descriptors of pain quality for dry stainless steel electrodes of varying diameters.

Finite Element model

The electrical potential and magnitude of the current density in the skin were calculated by a FE model simplifying the skin into four horizontal purely resistive layers. Several features of the skin were not included in the model. The low-resistance skin appendages such as sweat glands and hair follicles have been shown to induce a region of high current density in the proximity of the appendages [13]. Capacitance of the skin was not considered even though capacitive effects have been reported [23-25]. However, when mounting the electrodes, the skin may have been compressed or scratched, and this may especially for the smallest electrodes, have caused a compression of mainly the stratum corneum which may likely have lowered the capacitance. Non-linear behavior of the electrical properties [26] probably caused by conductive fluid extravasation driven by potential differences of the skin [27] was also neglected, although this may be dependent on the size of the electrode/the magnitude of the current density. Electrical properties for the electrodes and their interface with the skin was not considered as the current was modeled as an boundary condition of inward current at the area covered by the electrode. Despite these simplifications, the model provided resistance in the same order of magnitude as the measured impedances at high frequencies (10 kHz) for the large electrodes, FIG. 4. Obviously, any capacitive effects of the tissue are less pronounced at high frequencies applied through large electrodes. Capacitive effects are therefore likely a main the reason for the higher measured impedances at lower frequencies for the present dry, stainless steel electrodes.

### Branching Nerve Fiber Model

The numbers of simulated Aβ- and Aδ-fiber were selected so that the resulting nerve fiber density matched values reported in the literature. The density of Aβ-fiber endings has been reported to be 59.0±29.3 per mm² in the fingertip [15]. This density may differ from the density the Aβ-fibers in the volar forearm, however the same group did not find morphological differences for Aβ-fibers between the fingertip and the hairy skin of the leg [9]. The density of epidermal nerve endings has been assessed in several studies to establish reference values for diagnosing small fiber neuropathy [28]. A weighted average of the values listed in a recent review by Ebenezer et al. [10] shows an Aβ fiber density in the distal part of the leg of 12.8±2.0 nerve endings per mm, corresponding to density of 163±2.8 nerve fiber endings per mm² assuming isotropic horizontal directions.

The nerve fibers were modeled as branching with an equal probability of 10% at each node. This simple model of nerve branching resulted in denser branching when the nodes are closer spaced as in thin unmyelinated Aβ-fiber branches in the epidermis [28]. However, this is still a simplification of nerve Aβ-fibers, as they form a horizontal neural plexus in the upper part of dermis [28]. From the plexus, nerve branches penetrate vertically through the dermoepidermal junction and terminate in the vial layers of epidermis [10, 12]. The horizontal plexus may be organized as concentric horizontal circles [29]. The present model does not include such horizontal nerve plexuses. However, high current density in the epidermis where the Aβ-fibers terminate seems to be the key factor in the present model of thin fiber activation. Further branching may occur more proximal outside the modeled volume [30, 31].

Histological reports of myelinated Aβ-fiber innervating Merkel complexes reported distal fiber diameters of 3-4 μm [9, 15]. These measured distal thicknesses are significantly thinner than the more proximal main branch. In the present study, the thickness of the Aβ-fibers was adopted from a previous review [16]. Thicker nerve fibers were not considered in the present model as these fibers do not terminate superficially. The diameter of the Aβ-fibers was initiated at 4 μm and was modeled to gradually shrink by a factor of 0.9 at each branch point. As the fiber entered the epidermis the thickness was decreased to 1 μm in agreement with Reilly et al [17] who described unmyelinated epidermal nerve endings to have a thickness of 4-5 μm when entering the dermis.

### Perception Threshold

For vibration, excitation of a single afferent nerve is sufficient to evoke a sensory perception of the stimulus. However, by intraneural microstimulation, it has been shown that activation of a single neuron is not sufficient to evoke pain and touch except at the fingertips [18, 32]. For comparing simulated thresholds and measured thresholds in the present experiment, 3% of the simulated neurons need to be activated for the simulated perception thresholds to fit the measured perception thresholds.

The model showed that using electrodes with diameters less than 4 mm, M fibers had the lowest excitation threshold. This was further supported by the pain quality descriptors used by the subjects as stimulation through small electrodes was primarily described as ‘sharp’, ‘cutting’. For larger electrodes the most frequently used descriptors were ‘dull’, ‘sore’, ‘spreading’, and ‘radiating’. These pain descriptors are in accordance with Bromm and Meier [4] who reported ‘stabbing’, ‘sharp’, ‘hot’, and ‘troublesome’ for 1.2 mm intracutaneous electrodes and ‘pulsing’, ‘dull’, ‘penetrating’, and ‘annoying’ for superficial large electrode stimuli.

The excitation thresholds of the modeled Aδ-fibers had similar values as the experimentally measured perception threshold for electrodes smaller than 3 mm. The variation in the experimentally measured perception thresholds for larger electrodes was larger than the modeled excitation thresholds, indicating that the total variation in the sensory system is not fully explained in the model.

### Preferential Excitation of Nociceptors

Several studies have indicated that small diameter electrodes should be used when targeting nociceptors. Bromm and Meier [4] introduced an invasive intracutaneous electrical stimulation method. The pain quality reported by the subjects indicates preferential excitation of nociceptors. Nociceptive blink reflexes have been evoked by a 0.5 mm diameter cathode with a concentrically located anode. These reflexes could be almost abolished by topical application of local anesthesia indicating that they were mediated by thin fibers [7]. Furthermore, assessment of cortical evoked potential latencies indicate nerve fiber conduction velocity in the Aδ-fiber range for stimulation through small diameter electrodes [6]. The hypothesis that the magnitude of the current density was higher for small diameter electrodes was confirmed by the FE model. The branching nerve fiber model further indicated that Aδ-fibers were excited at lower currents than Aβ-fibers when small diameter fibers were utilized. These findings support the evidence that small diameter cutaneous surface electrodes to a larger degree activate nociceptors as long as the stratum corneum is not too thick.

### Applications

Several studies have utilized small diameter electrodes for cutaneous surface stimulation under the assumption that nociceptors are preferentially activated. An electrode consisting of numerous small cathodes have been used for induction of electrophysiological and perceptual correlates of long-term potentiation or long term depression [33-35]. Such strengthening of synapses are well-known from animal studies of the nociceptive system [36] but requires a heavy barrage of the nociceptive system (e.g. from direct nerve stimulation) to manifest so that the nociceptive barrage can ‘over-write’ the tactile input [37]. In animal studies such profound stimulation can be provided by direct stimulation of a sensory nerve. Long-term changes in the human nociceptive system after stimulation by large surface electrodes has not been reported perhaps due to a gate-control like occlusion of the nociceptive barrage by concurrent excitation of non-nociceptive afferents. Finally, itch can be inhibited by stimulation through an array of small diameter electrodes most likely mimicking scratching of the skin [5]. Electrical stimulation is also used when assessing the nociceptive withdrawal reflex in healthy volunteers and patients [38]. The evoked response is known to be composed of a mechanoreceptive RII component and a nociceptive RIII component [39]. Preferential activation of thinly myelinated afferents will therefore allow a more direct assessment of human spinal nociception. Although several studies have implicated a preferential activation nociceptors by stimulation of small area electrodes clinical validation e.g. in Wallenberg' s syndrome is still lacking.

The described study has demonstrated that high current densities at the dermoepidermal junction may results in preferential activation of Aδ-fibers instead of Aβ-fibers. This may be achieved by electrical stimulation through small area electrodes according to the present finite element model of the extracellular potential in combination with a model of the cutaneous afferent fibers based on stochastic fiber branching. Consequently, electrical stimulation through small area electrodes provides a method for activating the nociceptive system with minimal occlusion from non-nociceptive input.

Although the present invention has been described in connection with preferred embodiments, it is not intended to be limited to the specific form set forth herein. Rather, the scope of the present invention is limited only by the accompanying claims.

In this section, certain specific details of the disclosed embodiments are set forth for purposes of explanation rather than limitation, so as to provide a clear and thorough understanding of the present invention. However, it should be understood readily by those skilled in this art, that the present invention may be practiced in other embodiments which do not conform exactly to the details set forth herein, without departing significantly from the spirit and scope of this disclosure. Further, in this context, and for the purposes of brevity and clarity, detailed descriptions of well-known device, circuits and methodology have been omitted so as to avoid unnecessary detail and possible confusion.

In the claims, the term “comprising” does not exclude the presence of other elements or steps. Additionally, although individual features may be included in different claims, these may possibly be advantageously combined, and the inclusion in different claims does not imply that a combination of features is not feasible and/or advantageous. In addition, singular references do not exclude a plurality. Thus, references to “a”, “an”, “first”, “second” etc. do not preclude a plurality. Reference signs are included in the claims however the inclusion of the reference signs is only for clarity reasons and should not be construed as limiting the scope of the claims.

### LIST OF REFERENCES

1. D. Le Bars, M. Gozariu, S. W. Cadden, Pharmacological Reviews 53, 597 (2001).
2. E. A. Blair, J. Erlanger, Am J Physiol 106, 524 (1933).
3. P. D. Wall, Brain 101, 1 (1978).
4. B. Bromm, W. Meier, Methods and Findings in Experimental and Clinical Pharmacology 6, 405 (1984).
5. H. J. Nilsson, A. Levinsson, J. Schouenborg, Pain 71, 49 (1997).
6. K. Inui, T. D. Tran, M. Hoshiyama, R. Kakigi, Pain 96, 247 (2002).
7. H. Kaube, Z. Katsarava, T. Kaufer, H. C. Diener, J. Ellrich, Clinical Neurophysiology 111, 413 (2000).
8. D. R. Mcneal, Ieee Transactions on Biomedical Engineering 23, 329 (1976).
9. V. Provitera et al., Muscle & Nerve 35, 767 (2007).
10. G. J. Ebenezer, P. Hauer, C. Gibbons, J. C. McArthur, M. Polydefkis, Journal of Neuropathology and Experimental Neurology 66, 1059 (2007).
11. E. P. Gardner, J. H. Martin, T. M. Jessell, in Principles of Neural Science, E. R. Kandel, J. H. Schwartz, T. M. Jessell, Eds. (McGraw-Hill, 2000), chap. 22, pp. 430-450.
12. M. Hilliges, L. X. Wang, O. Johansson, Journal of Investigative Dermatology 104, 134 (1995).
13. N. Sha et al., Artificial Organs 32, 639 (2008).
14. P. J. Vauhkonen, M. Vauhkonen, T. Savolainen, J. P. Kaipio, Ieee Transactions on Biomedical Engineering 46, 1150 (1999).
15. M. Nolano et al., Annals of Neurology 54, 197 (2003).
16. A. Iggo, K. H. Andres, Annual Review of Neuroscience 5, 1 (1982).
17. D. M. Reilly et al., British Journal of Dermatology 137, 163 (1997).
18. R. S. Johansson, A. B. Vallbo, Journal of Physiology-London 297, 405 (1979).
19. K. Yoshida, A. Inmann, M. K. Haugland, IFESS 1999, Proceedings of the 4th Annual Conference of the International Functional Electrical Stimulation Society 267 (1999).
20. G. A. Gescheider, Psychophysics, The Fundamentals (Lawrence Erlbaum Associates, Inc., Publishers, Mahwah; NJ, 1997).
21. R. Melzack, Pain 1, 277 (1975).
22. A. M. Drewes et al., Clinical Journal of Pain 9, 80 (1993).
23. T. Yamamoto, Y. Yamamoto, Medical & Biological Engineering 14, 151 (1976).
24. A. Tavernier, M. Dierickx, M. Hinsenkamp, Bioelectrochemistry and Bioenergetics 30, 65 (1993).
25. S. Gabriel, R. W. Lau, C. Gabriel, Physics in Medicine and Biology 41, 2251 (1996).
26. T. Yamamoto, Y. Yamamoto, Medical & Biological Engineering & Computing 19, 302 (1981).
27. J. C. Weaver, Journal of Cellular Biochemistry 51, 426 (1993).
28. G. Lauria et al., European Journal of Neurology 12, 747 (2005).
29. M. Ishihara, R. Endo, M. R. P. Rivera, M. Mihara, Archives of Dermatological Research 294, 281 (2002).
30. C. Weidner, R. Schmidt, M. Schmelz, H. E. Torebjork, H. O. Handwerker, Journal of Physiology-London 547, 931 (2003).
31. Y. B. Peng, M. Ringkamp, J. N. Campbell, R. A. Meyer, J Neurophysiol 82, 1164 (1999).
32. J. Ochoa, E. Torebjork, Journal of Physiology-London 415, 583 (1989).
33. T. Klein, W. Magerl, H. C. Hopf, J. Sandkuhler, R. D. Treede, J. Neurosci. 24, 964 (2004).
34. J. A. Biurrun Manresa, C. D. Mørch, O. K. Andersen, European Journal of Pain In Press, Corrected Proof, (2010).
35. S. Rottmann, K. Jung, J. Ellrich, European Journal of Pain In Press, Corrected Proof, (2009).
36. R. R. Ji, T. Kohno, K. A. Moore, C. J. Woolf, Trends in Neurosciences 26, 696 (2003).
37. J. Sandkuhler, X. G. Liu, European Journal of Neuroscience 10, 2476 (1998).
38. O. K. Andersen, Acta Physiologica 189, 1 (2007).
39. J. C. Willer, Pain 3, 69 (1977).
40. J. Sandby-Moller, T. Poulsen, H. C. Wulf, Acta Dermato-Venereologica 83, 410 (2003).
41. S. Neerken, G. W. Lucassen, M. A. Bisschop, E. Lenderink, T. A. M. Nuijs, Journal of Biomedical Optics 9, 274 (2004).
42. B. D. Fornage, M. H. McGavran, M. Duvic, C. A. Waldron, Radiology 189, 69 (1993).
43. P. Krackowizer, E. Brenner, Phlebologie 37, 83 (2008).
44. C. Gabriel, S. Gabriel, E. Corthout, Physics in Medicine and Biology 41, 2231 (1996).
45. C. Gabriel, A. Peyman, E. H. Grant, Physics in Medicine and Biology 54, 4863 (2009).

What is claimed is:

1. A method for predicting afferent nerve fiber excitation from an electrical potential applied to a cutaneous tissue by an electrode, the method comprising
providing a model of propagation of electrical potentials in the cutaneous tissue,
providing a model of geometric distribution of afferent nerve fibers in the cutaneous tissue, wherein said model comprises a stochastic model of branching of afferent nerve fibers in the cutaneous tissue,
providing a geometric representation of the electrode in or on a surface of the cutaneous tissue, and
predicting afferent nerve fiber excitation from the electrical potential applied to the electrode by combining said model of propagation, said model of geometric distribution of afferent nerve fibers, and said geometric representation of the electrode.

2. The method according to claim 1, wherein the model of geometric distribution of afferent nerve fibers in the cutaneous tissue comprises specifying a probability that a nerve fiber will branch into a first and a second branching nerve fiber.

3. The method according to claim 2, wherein the model of geometric distribution of afferent nerve fibers in the cutaneous tissue comprises specifying a first stochastic direction towards a skin surface for the first branching fiber and a second stochastic direction towards a skin surface for the second branching nerve fiber.

4. The method according to claim 3, wherein said first and second directions towards a skin surface are determined by respective uniform distributions of azimuthal and horizontal angles towards the skin surface.

5. The method according to claim 1, wherein a location in the cutaneous tissue where a nerve fiber emerges is modelled as a random location within a specified area.

6. The method according to claim 1, wherein a cutaneous depth where a nerve fiber ends is modelled stochastically.

7. The method according to claim 1, wherein the model of propagation of electrical potentional in the cutaneous tissue comprises at least two different cutaneous layers with different electrical properties.

8. The method according to claim 7, wherein the model of propagation of electrical potentionals in the cutaneous tissue comprises modelling of different electrical properties of the hypodermis, dermis, epidermis, or stratum corneum.

9. The method according to claim 1, wherein the model of propagation of electrical potentials in the cutaneous tissue comprises a Finite Element model.

10. The method according to claim 9, wherein the Finite Element model is a two dimensional axial symmetrical Finite Element model.

11. The method according to claim 9, wherein the model of propagation of electrical potentials in the cutaneous tissue comprises a model of extracellular potentials.

12. The method according to claim 1, further comprising providing separate models of geometric distribution in the cutaneous tissue of a nociceptive nerve fiber and a non-nociceptive nerve fiber, with respective different stochastic parameters specified for said two types of nerve fibers.

13. The method according to claim 12, further comprising predicting separate non-nociceptive nerve fiber excitation and nociceptive nerve fiber excitation from the electrical potential applied to the electrode.

14. The method according to claim 12, further comprising providing separate models of distribution in the cutaneous tissue of Aβ nerve fibers and Aδ nerve fibers.

15. The method according to claim 1, wherein a membrane potential is calculated along points representing nodes of Ranvier on the nerve fibers.

16. The method according to claim 15, wherein a current equilibrium is calculated at each node of Ranvier according to Kirchoff s law.

17. A computer executable program code arranged to cause a computer device to perform the method according to claim 1.

18. A device arranged to perform the method according to claim 1.

19. A method for designing a cutaneous electrode comprising:
performing the method according to claim 1, and
changing a geometric design of the cutaneous electrode based on the predicted afferent nerve fiber excitation.

20. An electrode for cutaneous stimulation designed according to the method according to claim 19.
